(19) United States
(12) Patent Application Publication
(10) Pub. No.: US 2008/0214562 A1
(43) Pub. Date: Sep. 4, 2008

(54) Chemical Compounds

(76) Inventor: Kristjan Gudmundsson, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/995,231

(22) PCT Filed: Jul. 5, 2006
(86) PCT No.: PCT/US06/26239

§ 371 Date: Jan. 10, 2008

Related U.S. Application Data
(60) Provisional application No. 60/698,110, filed on Jul. 11, 2005.

(51) Int. Cl. A61K 31/496; C07D 221/04; A61K 31/436; A61P 31/18; C07D 295/00

(52) U.S. Cl. 514/253.04; 546/115; 514/302; 544/362

(57) ABSTRACT

The present invention provides compounds that demonstrate protective effects on target cells from HIV infection in a manner as to bind to a chemokine receptor, and which affect the binding of the natural ligand or chemokine to a receptor such as CXCR4 of a target cell.

### FIELD OF THE INVENTION

The present invention provides compounds that demonstrate protective effects on target cells from HIV infection in a manner as to bind to a chemokine receptor, and which affect the binding of the natural ligand or chemokine to a receptor such as CXCR4 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with the selective CXCR4 antagonist AMD-3100, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection asthma and allergic airway inflammation, Alzheimer's disease, and arthritis.

The present invention is directed to compounds that can act as agents that modulate chemokine receptor activity. Such chemokine receptors might include, but are not limited to, CCR1, CCR2, CCR3, CCR4, CCR5, CCR6, CCR7, CCR8, CXCR1, CXCR2, CXCR3, CXCR4, and CXCR5.

The present invention provides novel compounds that demonstrate protective effects on target cells from HIV infection in a manner as to bind specifically to the chemokine receptor, and which affect the binding of the natural ligand or chemokine to a receptor, such as CXCR4 of a target cell.

### SUMMARY OF THE INVENTION

The present invention includes compounds of formula (I):

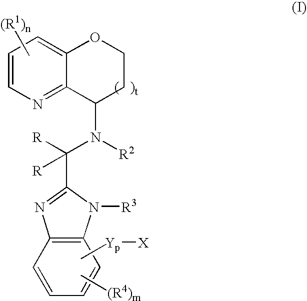

wherein:
t is 1, or 2;
each R independently is H, C₁-C₈ alkyl, C₂-C₆ alkenyl, C₂-C₆ alkynyl, C₁-C₈haloalkyl, C₃-C₇ cycloalkyl, —R^{a}Ay, —R^{a}OR⁵, or —R^{a}S(O)_{q}R⁵;
each R¹ independently is halogen, C₁-C₈haloalkyl, C₁-C₈ alkyl, C₂-C₆ alkenyl, C₂-C₆ alkynyl, C₃-C₇ cycloalkyl, C₃-C₇ cycloalkenyl, -Ay, —NHAy, -Het, —NHHet, —OR¹⁰, —OAy, —OHet,
R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, S(O)_{q}R¹⁰, S(O)_{q}Ay, cyano, nitro, or azido;
n is 0, 1, or 2;
R² is selected from a group consisting of H, C₁-C₈ alkyl, C₁-C₈haloalkyl, C₃-C₇ cycloalkyl, C₂-C₆ alkenyl, C₂-C₆ alkynyl, —R^{a}Ay, —R^{a}OR⁵, —R^{a}S(O)_{q}R⁵;
R³ is H, C₁-C₈ alkyl, C₁-C₈haloalkyl, C₃-C₇ cycloalkyl, C₂-C₆ alkenyl, C₂-C₆ alkynyl, —R^{a}Ay, —R^{a}OR⁵, or R^{a}S(O)_{q}R⁵;
each R⁴ independently is halogen, C₁-C₈haloalkyl, C₁-C₈ alkyl, C₂-C₆ alkenyl, C₂-C₆ alkynyl, C₃-C₇ cycloalkyl, C₃-C₇ cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, C₁-C₈ alkyl, C₂-C₆ alkenyl, C₂-C₆ alkynyl, C₃-C₇ cycloalkyl, or -Ay;
p is 0or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, R^{a}N(R¹⁰)₂, -AyN(R¹⁰)₂, —R^{a}AyN(R¹⁰)₂, AyR^{a}N(R¹⁰), R^{a}AyR^{a}N(R¹⁰)₂, R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷, or —R^{a}Het, provided that when p is 0 and X is —N(R¹⁰)₂, then R¹⁰ is not H;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each q independently is 0, 1, or 2;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and each Het independently represents a heterocyclyl or heteroaryl group optionally substituted with one or more of C₁-C₉ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or pharmaceutically acceptable derivatives thereof.

The present invention features a compound of formula (I) wherein t is 1 and all other substituents are as defined above or a pharmaceutically acceptable derivative thereof.

The present invention features a compound of formula (I) wherein R is H or C₁-C₈ alkyl and all other substituents are as defined above or a pharmaceutically acceptable derivative thereof.

The present invention features a compound of formula (I) wherein R is H and all other substituents are as defined above or a pharmaceutically acceptable derivative thereof.

The present invention features a compound of formula (I) wherein n is 0 and all other substituents are as defined above or a pharmaceutically acceptable derivative thereof.

The present invention features a compound of formula (I) wherein n is 1 and R¹ is halogen, C₁-C₈haloalkyl, C₁-C₈ alkyl, OR¹⁰, NR⁶R⁷, CO₂R¹⁰, CONR⁶R⁷, or cyano and all other substituents are as defined above or a pharmaceutically acceptable derivative thereof.

The present invention features a compound of formula (I) wherein R² is H, C₁-C₈ alkyl, C₁-C₈haloalkyl, or C₃-C₇ cycloalkyl and all other substituents are as defined above or a pharmaceutically acceptable derivative thereof.

The present invention features a compound of formula (I) wherein R² is C₁-C₈ alkyl, C₁-C₈haloalkyl, or C₃-C₇ cycloalkyl and all other substituents are as defined above or a pharmaceutically acceptable derivative thereof.

The present invention features a compound of formula (I) wherein R³ is H, C₁-C₈ alkyl, C₁-C₈haloalkyl, C₃-C₇ cycloalkyl, C₂-C₆ alkenyl, or C₂-C₆ alkynyl and all other substituents are as defined above or a pharmaceutically acceptable derivative thereof.

The present invention features a compound of formula (I) wherein R³ is H, C₁-C₈ alkyl, C₁-C₈haloalkyl, or C₃-C₇ cycloalkyl and all other substituents are as defined above or a pharmaceutically acceptable derivative thereof. The present invention also features a compound of formula (I) wherein R³ is H or C₁-C₈ alkyl and all other substituents are as defined above or a pharmaceutically acceptable derivative thereof.

The present invention features a compound of formula (I) wherein m is 0 and all other substituents are as defined above or a pharmaceutically acceptable derivative thereof.

The present invention features a compound of formula (I) wherein m is 1 or 2 and R⁴ is one or more of halogen, C₁-C₈haloalkyl, C₁-C₈ alkyl, OR¹⁰, NR⁶R⁷, CO₂R¹⁰, CONR⁶R⁷, or cyano and all other substituents are as defined above or a pharmaceutically acceptable derivative thereof.

The present invention features a compound of formula (I) wherein m is 1 and all other substituents are as defined above or a pharmaceutically acceptable derivative thereof.

The present invention features a compound of formula (I) wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof.

The present invention features a compound of formula (I) wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂. The present invention also features a compound of formula (I) wherein p is 0, X is selected from -Het or -HetN(R¹⁰)₂ and all other substituents are as defined above or a pharmaceutically acceptable derivative thereof.

The present invention features a compound of formula (I) wherein p is 1; Y is —N(R¹⁰)—, —O—, —S—, —CONR¹⁰—, —NR¹⁰CO—, or —S(O)_{q}NR¹⁰—; X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof.

The present invention features a compound of formula (I) wherein Het is a 4-, 5-, or 6-membered heterocyclyl or heteroaryl group. The present invention features a compound of formula (I) wherein Het is piperidine, piperazine, azetidine, pyrrolidine, imidazole, pyridine, and the like and all other substituents are as defined above or a pharmaceutically acceptable derivative thereof.

The present invention features a compound of formula (I) wherein Het is C₁-C₈ alkyl substituted piperazine and all other substituents are as defined above or a pharmaceutically acceptable derivative thereof.

The present invention features a compound wherein the substituent —Yₚ—X is located on the depicted benzimidazole ring as in formula (I-A):

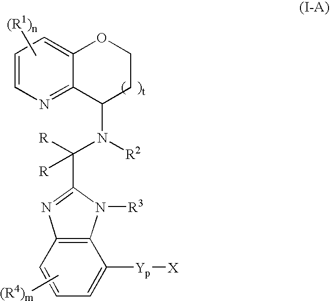

wherein:
t is 1 or 2;
each R independently is H, C₁-C₈ alkyl, C₂-C₆ alkenyl, C₂-C₆ alkynyl, C₁-C₈haloalkyl, C₃-C₇ cycloalkyl, —R^{a}Ay, —R^{a}OR⁵, or —R^{a}S(O)_{q}R⁵;
each R¹ independently is halogen, C₁-C₈haloalkyl, C₁-C₈ alkyl, C₂-C₆ alkenyl, C₂-C₆ alkynyl, C₃-C₇ cycloalkyl, C₃-C₇ cycloalkenyl, -Ay, —NHAy, -Het, —NHHet, —OR¹⁰, —OAy, —OHet, R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, C₁-C₈ alkyl, C₁-C₈haloalkyl, C₃-C₇ cycloalkyl, C₂-C₆ alkenyl, C₂-C₆ alkynyl, —R^{a}Ay, —R^{a}OR⁵, —R^{a}S(O)_{q}R⁵;
R³ is H, alkyl, C₁-C₈haloalkyl, C₃-C₇ cycloalkyl, C₂-C₆ alkenyl, C₂-C₆ alkynyl, —R^{a}Ay, —R^{a}OR⁵, or —R^{a}S(O)_{q}R⁵;
each R⁴ independently is halogen, C₁-C₈haloalkyl, C₁-C₈ alkyl, C₂-C₆ alkenyl, C₂-C₆ alkynyl, C₃-C₇ cycloalkyl, C₃-C₇ cycloalkenyl, -Ay, —NHAy, -Het, —NHHet, —OR¹⁰, —OAy, —OHet, R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, C₁-C₈ alkyl, C₂-C₆ alkenyl, C₂-C₆ alkynyl, C₃-C₇ cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, AyR^{a}N(R¹⁰)₂, R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}Cycloalkyl, —R^{a}OH, —R^{a}OR⁵, —R^{a}NR⁶R⁷, or —R^{a}Het, provided that when p is 0 and X is —N(R¹⁰)₂, then R¹⁰ is not H;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each q independently is 0, 1, or 2;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or pharmaceutically acceptable derivatives thereof.

Another aspect of the invention includes compounds of formula (I-B):

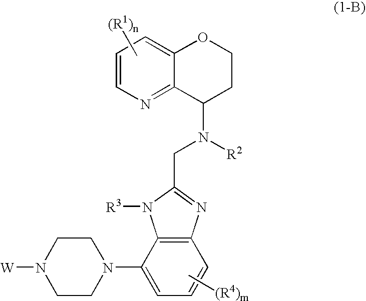

wherein R¹ is halogen, C₁-C₈haloalkyl, C₁-C₈ alkyl, OR¹⁰, NR⁶R⁷, CO₂R¹⁰, CONR⁶R⁷, or cyano;
n is 0, 1; or 2;
R² is H, C₁-C₈ alkyl, C₁-C₈haloalkyl or C₃-C₇cycloalkyl;
R³ is H, C₁-C₈ alkyl, C₁-C₈haloalkyl, C₃-C₇ cycloalkyl, C₂-C₆ alkenyl, or C₂-C₆ alkynyl;
each R^{a} independently is C₁-C₈ alkylene, C₃-C₇ cycloalkylene, C₂-C₆ alkenylene, C₃-C₇ cycloalkenylene, or C₂-C₆ alkynylene;
m is 0, 1, or 2;
R⁴ is one or more of halogen, C₁-C₈haloalkyl, C₁-C₈ alkyl, OR¹⁰, NR³R⁷, CO₂R¹⁰, CONR⁶R⁷, or cyano;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}Cycloalkyl, —R^{a}OH, —R^{a}OR⁵, —R^{a}NR⁶R⁷, or —R^{a}Het; each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each R^{a} independently is C₁-C₈ alkylene, C₃-C₇ cycloalkylene, C₂-C₆ alkenylene, C₃-C₇ cycloalkenylene, or C₂-C₆ alkynylene;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and
W is H or C₁-C₈ alkyl;
or pharmaceutically acceptable derivatives thereof.

Another aspect of the invention includes compounds of formula (I-C):

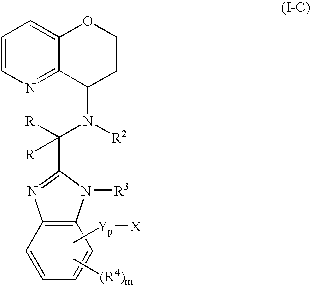

wherein each R is H or C₁-C₈ alkyl;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
m is 0, 1, or 2;
R⁴ is one or more of halogen, C₁-C₈haloalkyl, C₁-C₈ alkyl, OR¹⁰, NR⁶R⁷, CO₂R¹⁰, CONR⁶R⁷, or cyano;
p is 0 or 1;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂;

### Y is —N(R¹⁰)—, —O—, —S—, —CONR¹⁰—, —NR¹⁰CO—, or —S(O)_{q}NR¹⁰—;

each q independently is 0, 1, or 2;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷, or —R^{a}Het;
each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each R^{a} independently is C₁-C₈ alkylene, C₃-C₇ cycloalkylene, C₂-C₆ alkenylene, C₃-C₇ cycloalkenylene, or C₂-C₆ alkynylene;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or pharmaceutically acceptable derivatives thereof.

Another aspect of the invention includes compounds of formula (I-D):

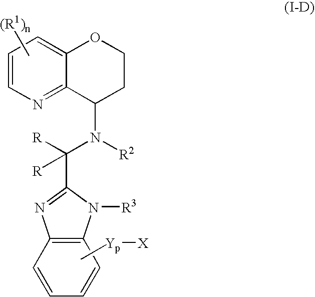

wherein each R is H or C₁-C₈ alkyl;
R¹ is halogen, C₁-C₈haloalkyl, C₁-C₈ alkyl, OR¹⁰, NR⁶R⁷, CO₂R¹⁰, CONR⁶R⁷, or cyano;
n is 0, 1; or 2;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
p is 0 or 1;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂;

### Y is —N(R¹⁰)—, —O—, —S—, —CONR¹⁰—, —NR¹⁰CO—, or —S(O)_{q}NR¹⁰—;

each q independently is 0, 1, or 2;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷, or —R^{a}Het; each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or pharmaceutically acceptable derivatives thereof.

Another aspect of the invention includes compounds of formula (I-E)

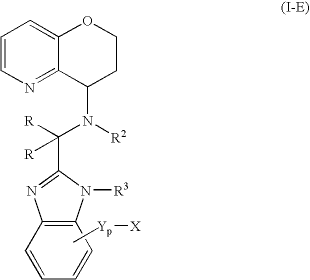

wherein each R is H or C₁-C₈ alkyl;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
p is 0 or 1;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂;

### Y is —N(R¹⁰)—, —O—, —S—, —CONR¹⁰—, —NR¹⁰CO—, or —S(O)_{q}NR¹⁰)—;

each q independently is 0, 1, or 2;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆alkynyl, C₃-C₇ cycloalkenyl, —R^{a}cycloalkyl, —R^{a}OH, —R^{a}OR⁵, —R^{a}NR⁶R⁷, or —R^{a}Het; each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R¹⁰H, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino; and
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or pharmaceutically acceptable derivatives thereof.

The present invention features a compound selected from the group consisting of:
N-(2-{[3,4-dihydro-2H-pyrano[3,2-b]pyridin-4-yl(methyl)amino]methyl}-1H-benzimidazol-4-yl)-N,N,N-trimethyl-1,2-ethanediamine;
N-methyl-N-{[4-(1-piperazinyl)-1H-benzimidazol-2-yl]methyl}-3,4-dihydro-2H-pyrano[3,2-b]pyridin-4-amine;
N-methyl-N-{[4-(4-methyl-1-piperazinyl)-1H-benzimidazol-2-yl]methyl}-3,4-dihydro-2H-pyrano[3,2-b]pyridin-4-amine; and
N-methyl-N-{[1-methyl-7-(4-methyl-1-piperazinyl)-1H-benzimidazol-2-yl]methyl}-3,4-dihydro-2H-pyrano[3,2-b]pyridin-4-amine;
   or pharmaceutically acceptable derivatives thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment or prophylaxis of diseases and conditions modulated by a chemokine receptor, such as for example, CXCR4.

One aspect of the present invention includes one or more compounds of the present invention for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. In one embodiment, the condition or disease is HIV infection, rheumatoid arthritis, inflammation, or cancer.

One aspect of the present invention includes the use of one or more compounds of the present invention in the manufacture of a medicament for use in the treatment or prophylaxis of a condition or disease modulated by a chemokine receptor. Preferably the chemokine receptor is CXCR⁴.

One aspect of the present invention includes use of one or more compounds of the present invention in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection, rheumatoid arthritis, inflammation, or cancer.

One aspect of the present invention includes a method for the treatment or prophylaxis of a condition or disease modulated by a chemokine receptor comprising the administration of one or more compounds of the present invention. Preferably the chemokine receptor is CXCR⁴.

One aspect of the present invention includes a method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment or prophylaxis of HIV infection, rheumatoid arthritis, inflammation, or cancer comprising the administration of one or more compounds of the present invention.

### DETAILED DESCRIPTION OF THE INVENTION

Terms are used within their accepted meanings. The following definitions are meant to clarify, but not limit, the terms defined.

As used herein the term “alkyl” alone or in combination with any other term, refers to a straight or branched chain hydrocarbon, containing the specified number of carbon atoms. Unless specified otherwise, the alkyl group preferably has from one to twelve carbon atoms. Examples of “alkyl” as used herein include, but are not limited to, methyl, ethyl, propyl, isopropyl, isobutyl, n-butyl, tert-butyl, sec-butyl, isopentyl, n-pentyl, n-hexyl, and the like.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” alone or in combination with other terms, refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds, which may occur at any stable point along the chain. Examples include, but are not limited to, ethynyl, propynyl, butynyl, pentynyl, and the like.

As used herein, the term “alkylene” refers to a straight or branched chain divalent hydrocarbon radical, preferably having from one to ten carbon atoms, unless otherwise specified. Examples of “alkylene” as used herein include, but are not limited to, methylene, ethylene, n-propylene, n-butylene, and the like.

As used herein, the term “alkenylene” refers to a straight or branched chain divalent hydrocarbon radical, preferably having from two to ten carbon atoms, unless otherwise specified, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from two to ten carbon atoms, unless otherwise specified, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Unless otherwise indicated, cycloalkyl is composed of three to seven carbon atoms. Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “heterocycle”, “heterocyclic” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. In one embodiment, the heteroatom is N.

Preferably the heterocyclyl ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, and tetrahydrothiophene. When the heterocyclic ring has substituents, it is understood that the substituents may be attached to any atom in the ring, whether a heteroatom or a carbon atom, provided that a stable chemical structure results. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino.

As used herein, the term “aryl” refers to an optionally substituted carbocyclic aromatic moiety (such as phenyl or napthyl) containing the specified number of carbon atoms, preferably 6-14 carbon atoms or 6-10 carbon atoms. The term aryl also refers to optionally substituted ring systems, for example anthracene, phenanthrene, or naphthalene ring systems. Examples of “aryl” groups include, but are not limited to, phenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. In one embodiment the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined. Examples of suitable alkoxy radicals include, but are not limited to methoxy, ethoxy, propoxy, isopropoxy, n-butoxy, isobutoxy, sec-butoxy, tert-butoxy, and the like.

As used herein the term “cycloalkoxy” refers to a group —OR′, where R′ is cycloalkyl as defined.

As used herein the term “alkoxycarbonyl” refers to groups such as:

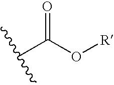

where the R′ represents an alkyl group as herein defined.

As used herein the term “aryloxycarbonyl” refers to groups such as:

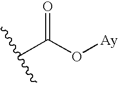

where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached.

As used herein the term “amide” refers to a group —C(O)NR′R″, where R′ and R″ independently represent H, alkyl, alkenyl, alkynyl, cycloalkyl, heterocyclyl, aryl, or heteroaryl.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for modifications which are encompassed within the scope of the appended claims.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process.

Compounds of the present invention may exist in unsolvated forms as well as solvated forms, including hydrated forms. Solvated forms and unsolvated forms are encompassed within the scope of the present invention. Compounds of the present invention may exist in a mixture of forms and/or solvates or as a mixture of amorphous material and one or more forms and/or solvates. In general, all physical forms are intended to be within the scope of the present invention. Forms may be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of the present invention, or a salt or other pharmaceutically acceptable derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. In one embodiment, the solvent used is water.

A “pharmaceutically acceptable derivative” means any pharmaceutically acceptable salt, ester, salt of an ester, ether, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators.

In one aspect of the present invention, the compounds demonstrate protective effects against HIV infection by inhibiting binding of HIV to a chemokine receptor such as CXCR4 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases such as rheumatoid arthritis, psoriatic arthritis, systemic lupus erythematosus, myastenia gravis, juvenile onset diabetes; glomerulonephritis, autoimmune throiditis, graft rejection, including allograft rejection or graft-versus-host disease; inflammatory bowel diseases, such as Crohn's disease and ulcerative colitus; spondyloarthropathies; scleroderma; psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis); eoosinophilic myotis, eosinophilic fasciitis; and cancers.

The compounds according to the invention may also be used in adjuvant therapy in the treatment of HIV infections or HIV-associated symptoms or effects, for example Kaposi's sarcoma.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment or prophylaxis of any of the aforementioned diseases or conditions.

Reference herein to treatment extends to prophylaxis as well as the treatment of established conditions, disorders and infections, symptoms thereof, and associated. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

For use in therapy, therapeutically effective amounts of a compound of the present invention, as well as salts, solvates, or other pharmaceutically acceptable derivatives thereof, may be administered as the raw chemical. Additionally, the active ingredient may be presented as a pharmaceutical composition.

Accordingly, the invention further provides pharmaceutical compositions that include effective amounts of compounds of the present invention or salts, solvates, or other pharmaceutically acceptable derivatives thereof, and one or more pharmaceutically acceptable carriers, diluents, or excipients. The compounds of the present invention and salts, solvates, and pharmaceutically acceptable derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention or salts, solvates, or other pharmaceutically acceptable derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from frailty, generally, should be in the range of 0.001 to 100 mg/kg body weight of recipient (mammal) per day. More usually the effective amount should be in the range of 0.001 to 1 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or pharmaceutically acceptable derivative thereof, may be determined as a proportion of the effective amount of a compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the present invention, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention or their salts, solvates, or other pharmaceutically acceptable derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention or salts, solvates, or other pharmaceutically acceptable derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention or their salts, solvates, or other pharmaceutically acceptable derivatives thereof, may be employed alone or in combination with other therapeutic agents. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention or salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, amprenavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870, 180 and similar agents;

Budding inhibitors such as PA-344 and PA-457, and similar agents; and

Other CXCR4 and/or CCR5 inhibitors such as Sch-C, Sch-D, TAK779, UK 427,857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

It should be understood that in addition to the ingredients particularly mentioned above the pharmaceutical compositions of this invention may include other agents conventional in the art having regard to the type of pharmaceutical composition in question, for example, those suitable for oral administration may include such further agents as sweeteners, thickeners and flavoring agents.

The compounds of the present invention may be prepared according to the following reactions schemes and examples, or modifications thereof using readily available starting materials, reagents and conventional synthesis procedures. In these reactions, it is also possible to make use of variants which are known to those of ordinary skill in the art.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention

Those skilled in the art will recognize if a stereocenter exists in compounds of the present invention. Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

### Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
RT (room temperature); h (hours);
min (minutes); TLC (thin layer chromatography);
mp (melting point); RP (reverse phase);
Tᵣ (retention time); TFA (trifluoroacetic acid);
TEA (triethylamine); THF (tetrahydrofuran);
TFAA (trifluoroacetic anhydride); CD₃OD (deuterated methanol);
CDCl₃ (deuterated chloroform); DMSO (dimethylsulfoxide);
SiO₂ (silica); atm (atmosphere);
EtOAc (ethyl acetate); CHCl₃ (chloroform);
HCl (hydrochloric acid); Ac (acetyl);
DMF (N,N-dimethylformamide); Me (methyl);
Cs₂CO₃ (cesium carbonate); EtOH (ethanol);
Et (ethyl); tBu (tert-butyl);
MeOH (methanol); p-TsOH (p-toluenesulfonic acid);
EDC (1-(3-dimethylaminopropyl)-3-ethylcarbodiimide hydrochloride);
HOBT (1-hydroxybenzotriazole);
BOPCI (bis(2-oxo-3-oxazolildinyl)phosphinic chloride);
HBTU (O-benzotriazol-1-yl-N,N,N′,N′-tetramethyluronium hexafluorophosphate);
MP-TsOH (polystyrene resin bound equivalent of p-TsOH from Argonaut Technologies).

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectra were acquired in CDCl₃ using a Bomem Chiral RTM VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R) — or (S)— configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality12 (2000) 172; and Gaussian 98, Revision A.11.4, M. J. Frisch et al., Gaussian, Inc., Pittsburgh Pa., 2002.

Compounds of formula (I) where all variables are as defined herein, and specifically wherein t is 1 and each R is hydrogen, can be prepared according to Scheme 1. Compounds of formula (I) wherein t is 2 can be made in a similar fashion as would be evident to one of skill in the art.

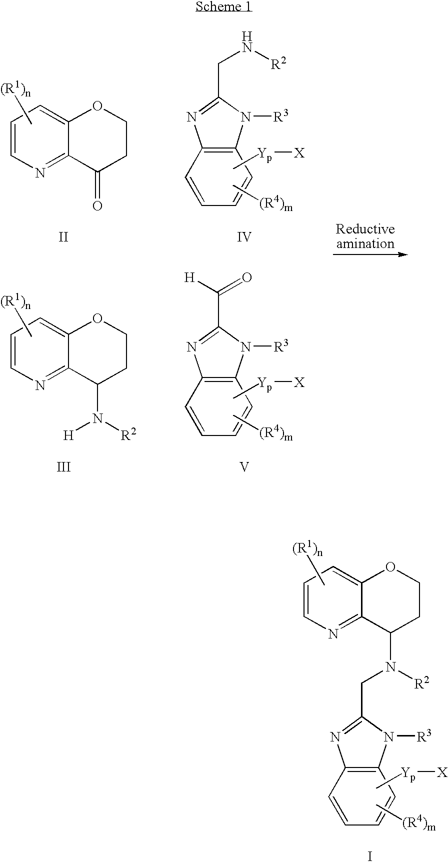

More specifically, compounds of formula (I) can be prepared by reacting a compound of formula (II) with a compound (IV) or alternatively reacting a compound of formula (III) with a compound of formula (V) under reductive conditions. The reductive amination can be carried out by treating the compound of formula (II) or (III) with a compound of formula (IV) or (V), respectively, in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally, the reaction can be run in presence of acid, such as acetic acid and the like.

Compounds of formula (II) can be prepared as described in the literature (J. Org. Chem., 2003, 68, 3546, WO2002022600; US2004019058 herein incorporated by reference with regard to such synthesis). Compounds of formula (II) can also be prepared from 3,4-dihydro-2H-pyrano[3,2-b]pyridin-4-yl acetate (Heterocycles, 1979, 12, 493 herein incorporated by reference with regard to such synthesis) by deprotection of the acetyl protected alcohol followed by oxidation. Compounds of formula (III) can be prepared by reductive amination of compound of formula (II) using processes well known to those skilled in the art of organic synthesis. Compounds of formula (IV) and (V) can be prepared by methods similar to those described in the literature (Tet. Lett. 1998, 39, 7467-7470; WO 02/092575; WO03/053344; WO03/106430; Science of Synthesis 2002, 12,529-612; each incorporated by reference with regard to such synthesis).

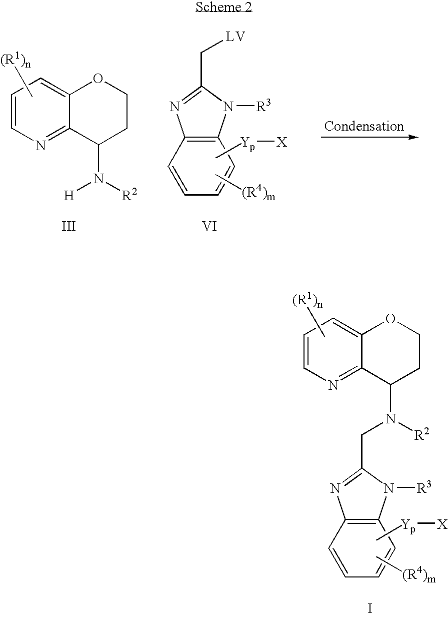

Compounds of formula (I) can be prepared by reaction of a compound of formula (III) with a compound of formula (VI) where LV is a leaving group (e.g., halogen, mesylate, tosylate) and all other variables are as defined herein, as outlined in Scheme 2. This condensation is typically carried out in a suitable solvent optionally in the presence of a base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, and the like. The reaction can be carried out at room temperature or optionally heated to 30-200° C. A catalyst, such as potassium iodide, tertbutylammonium iodide, or the like, can optionally be added to the reaction mixture.

Compounds of formula (I) can be prepared as outlined in Scheme 3, where Z is a suitable protecting group and all other variables are as defined in connection with compound of formula (I).

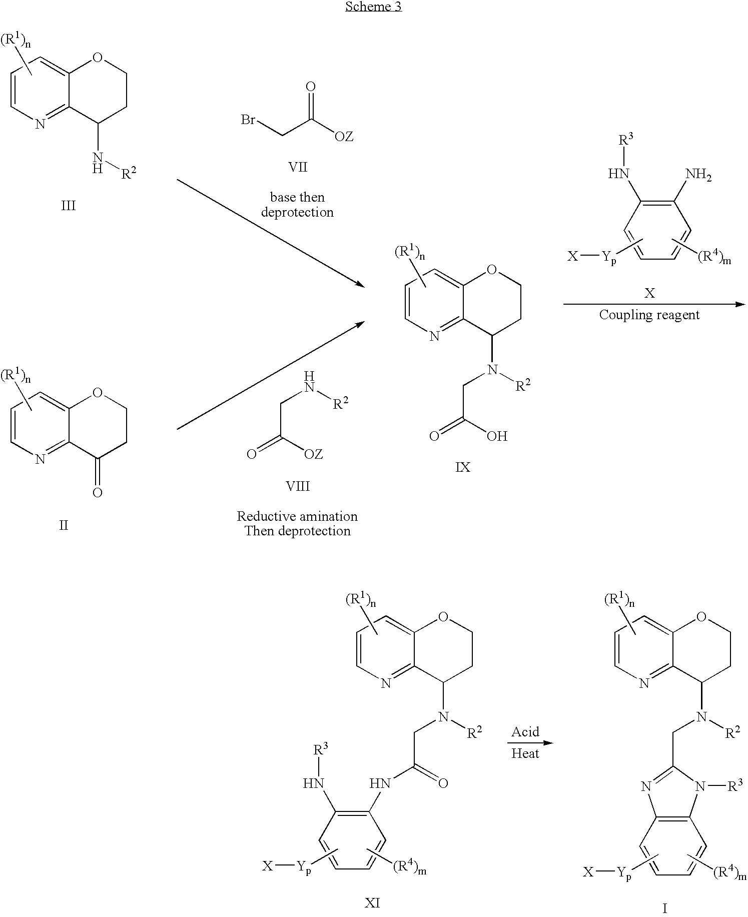

Compounds of formula (I) can be prepared by treatment of a compound of formula (XI) under acidic conditions optionally with heating. The reaction can be carried out by treating the compound of formula (XI) with a suitable acid optionally in the presence of an inert solvent, such as but not limited to tetrahydrofuran, acetonitrile, toluene, and the like. The reaction may be heated to 50-200° C. or performed at ambient temperature. Suitable acids include acetic acid, trifluoroacetic acid, hydrochloric acid, and the like. The reaction can be carried out using the acid as a solvent.

More specifically, as illustrated below, compounds of formula (XI) can be prepared by coupling of a compound of formula (X) with a compound of formula (IX). This coupling can be carried out using a variety of coupling reagents well known to those skilled in the art of organic synthesis (e.g., EDC, HOBt/HBTu; BOPCI). The reaction can be carried out with heating or at ambient temperature. Suitable solvents for this reaction include acetonitrile, tetrahydrofuran, and the like. Compounds of formula (X) can be prepared by methods known in the literature. Compounds of formula (IX) can be prepared from a compound of formula (II) and a protected glycine derivative (VIII) by reductive amination, followed by deprotection. Alternatively compound of formula (IX) can be prepared from compound of formula (III) and compound of formula (VII) via methods well known to those skilled in the art of organic synthesis.

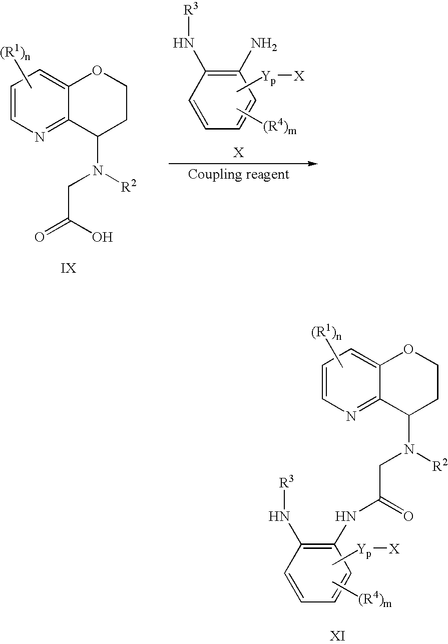

A compound of formula I-A wherein each R is H; R³ is H; p is 0; X is a substituted piperazine; and all other variables are as defined herein, can be prepared according to Scheme 4 where W is alkyl or a suitable protecting group. Compounds of formula I-B may be made in a similar manner.

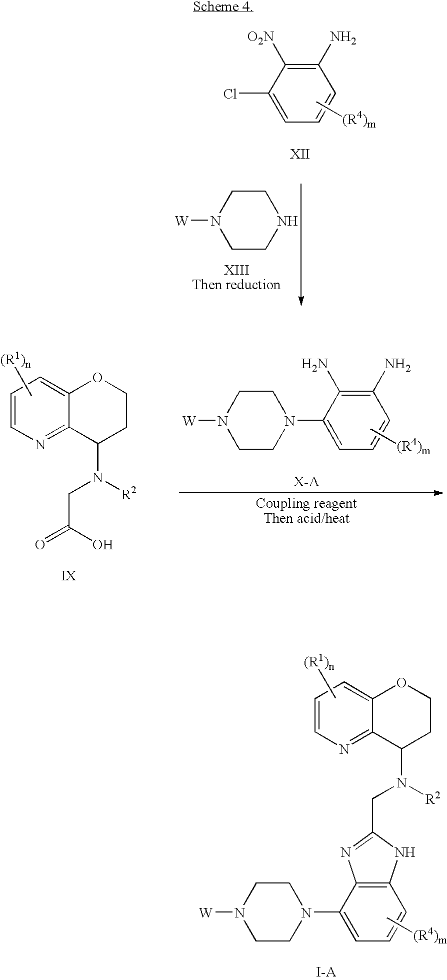

Generally the process of preparing compound of formula I-A where X is a substituted piperazine, R³ is H and all other variables are as defined herein above includes the steps of:
a) Reacting a compound of formula (XII) with a compound of formula (XIII), followed by reduction to form a compound of formula (X-A); and
b) Coupling a compound of formula (X-A) with a compound of formula (IX) and treating the coupled product with acid and heat to form a compound of formula I-A.

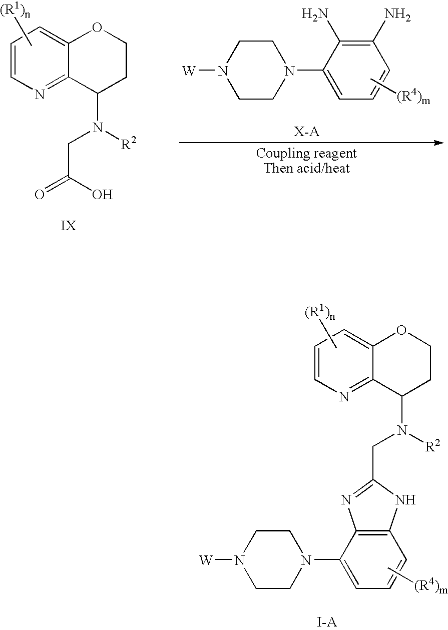

More specifically compounds of formula (I-A) can be prepared by coupling of compound of formula (X-A) and compound of formula (IX) followed by treatment with acid. Typical coupling reagents include EDC, HOBt/HBTu and BOPCI. Compounds of formula (I-A) can be prepared by treatment of the intermediate amide under acidic conditions optionally with heating. The reaction can also be carried out by treatment with a suitable acid optionally in the presence of an inert solvent. Suitable acids include acetic acid, trifluoroacetic acid, hydrochloric acid, and the like. Suitable solvents for this reaction include acetonitrile, tetrahydrofuran, and the like. The reaction may be heated to 50-200° C. or performed at ambient temperature. The reaction can be carried out using the acid as a solvent. Other suitable solvents include toluene, and the like. Compounds of formula (IX) can be prepared as described previously.

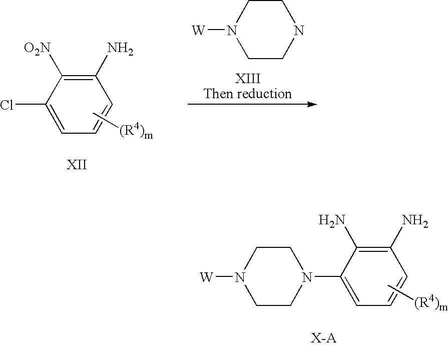

Compound of formula (X-A) can be prepared from a compound of formula (XII) and a compound of formula (XIII) by condensation optionally in the presence of solvent and optionally with heating or in a microwave, followed by reduction. Compounds of formula (XII) and (XIII) are readily commercially available or can be prepared by conditions well known to those skilled in the art of organic chemistry.

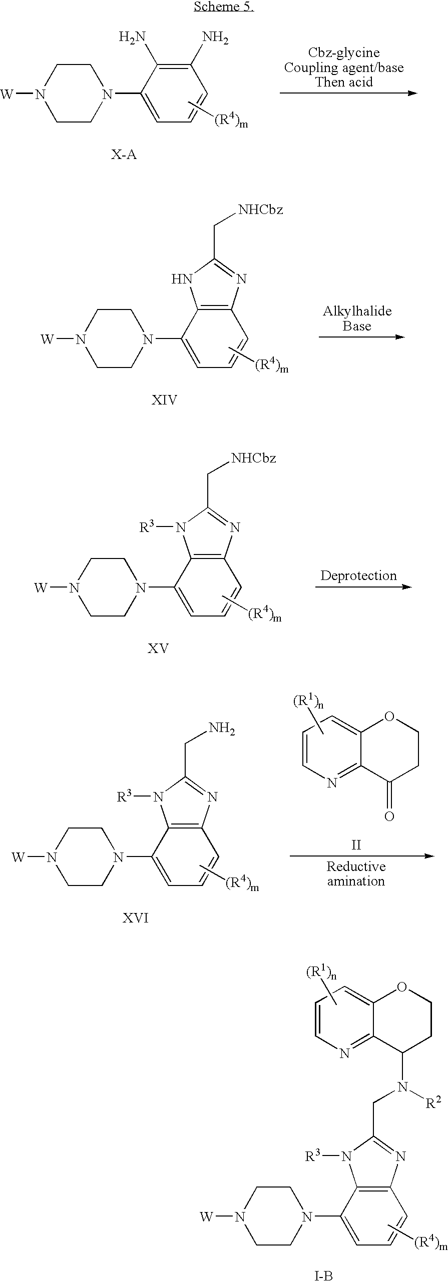

Generally the process of preparing compounds of formula I-B where R³ is alkyl, W is alkyl or a suitable protecting group, and all other variables are as defined hereinabove include the steps of:
a) Preparing a compound of formula (XIV) from a compound of formula (X-A) and protected glycine;
b) Preparing a compound of formula (XV) from a compound of formula (XIV);
c) Preparing a compound for formula (XVI) from a compound of formula (XV); and
d) Reacting a compound of formula (II) with compound of formula (XVI) to form compound of formula (I-B).

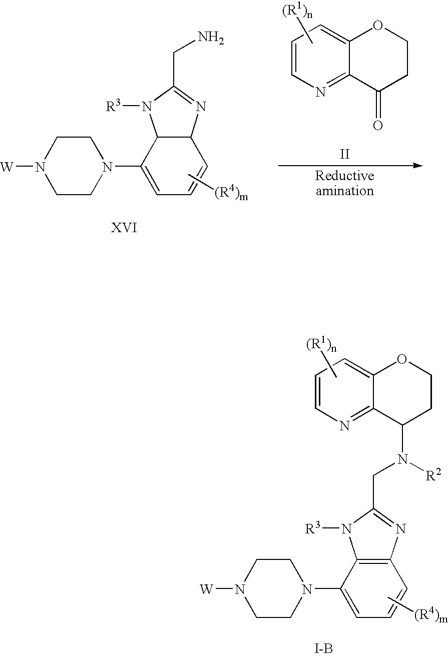

More specifically a compound of formula (I-B) can be prepared from a compound of formula (XVI) and a compound of formula (II) via reductive amination. The reductive amination can be carried out by treating the compound of formula (II) with a compound of formula (XVI) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

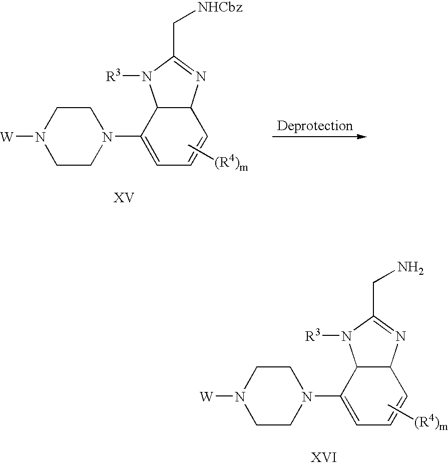

More specifically a compound of formula (XVI) can be prepared from a compound of formula (XV) by deprotection. For deprotection of Cbz protecting groups catalytic reduction is a suitable deprotection method. Suitable catalysts include Pd/C and the like. Suitable solvents include alcohols and the like.

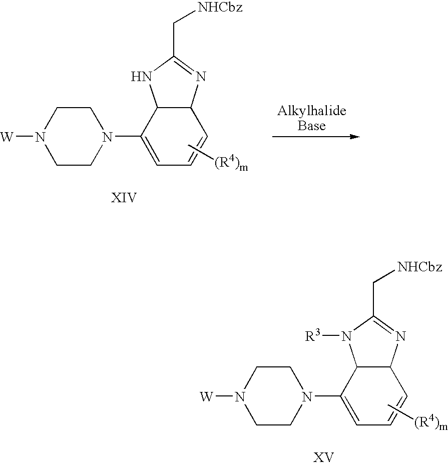

More specifically a compound of formula (XV) can be prepared from a compound of formula (XIV). Treatment of a compound of formula (XIV) with a suitable alkylhalide in a solvent, optionally with heating and optionally in the presence of base gives compound of formula (XV) as one of the obtained isomers. Suitable alkylhalides include methyliodide, ethyliodide and the like. Suitable solvents include dimethylformamide, dimethylsulfoxide, N-methylpyrrolidinone, nitromethane, acetonitrile and the like. Suitable bases include potassium carbonate, cesium carbonate, sodium hydride and the like. Reaction can optionally be heated between 20-200° C. or carried out in a microwave.

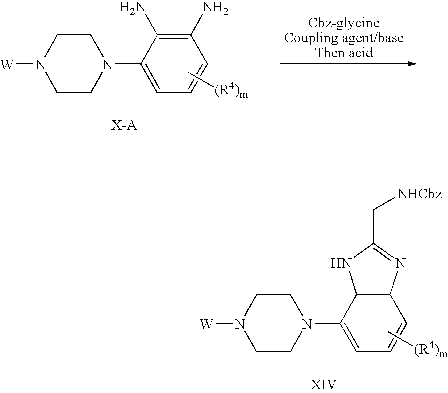

More specifically a compound of formula (XIV) can be prepared from a compound of formula (X-A). Treatment of a compound of formula (X-A) with Cbz-glycine and a suitable coupling agent (EDC, HOBt/HBTu and BOPCI) followed by treatment of the resulting amide under acidic conditions optionally with heating. The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

As is evident to one skilled in the art of organic synthesis an alternative in of preparing a compound of formula (XV) would be to use a compound of formula (XVII) as a stating material and upon ring closure obtain a benzimidazole isomer, as shown below. Treatment of a compound of formula (X-A) with Cbz-glycine and a suitable coupling agent (EDC, HOBt/HBTu and BOPCI) followed by treatment of the resulting amide under acidic conditions optionally with heating. The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

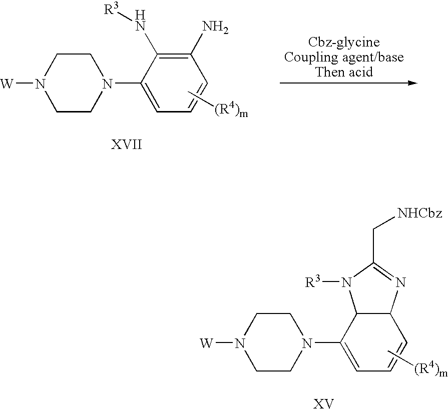

### EXAMPLES

### Example 1

### N-(2-{[3,4-dihydro-2H-pyrano[3,2-b]pyridin-4-yl(methyl)amino]methyl}-1H-benzimidazol-4-yl)-N,N,N-trimethyl-1,2-ethanediamine

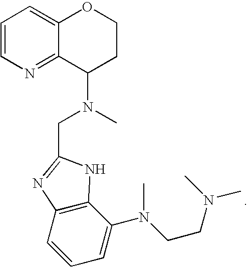

### A) Phenylmethyl N-(3,4-dihydro-2H-pyrano[3,2-b]pyridin-4-yl)glycinate

To a solution of 2,3-dihydro-4H-pyrano[3,2-b]pyridin-4-one (100 mg, 0.671 mmol) in 1,2 dichloroethane (3 mL) was added glycine benzyl ester hydrochloride (139 mg, 0.689 mmol) and acetic acid (48 μL, 0.839 mmol), respectively. Sodium triacetoxyborohydride (284 mg, 1.34 mmol) was added and the reaction mixture was stirred at room temperature overnight. The reaction mixture was diluted with dichloromethane and washed with saturated aqueous sodium bicarbonate solution. The organic layer was washed with brine then dried over sodium sulfate. Filtration and concentration provided Phenylmethyl N-(3,4-dihydro-2H-pyrano[3,2-b]pyridin-4-yl)glycinate (240 mg, 120%, possibly contaminated with salts) as a brownish oil.

¹H NMR (400 MHz, CDCl₃) δ 8.13 (m, 1H), 7.36-7.32 (m, 5H), 7.17-7.05 (m, 2H), 6.00 (br, 1H), 4.37-3.88 (m, 5H), 3.65 (m, 2H), 2.30-2.11 (m, 2H); MS m/z 299 (M+H)⁺.

### B) Phenylmethyl N-(3,4-dihydro-2H-pyrano[3,2-b]pyridin-4-yl)-N-methylglycinate

To a solution of phenylmethyl N-(3,4-dihydro-2H-pyrano[3,2-b]pyridin-4-yl)glycinate (480 mg, 1.61 mmol) in 1,2 dichloroethane (10 mL) was added formaldehyde (196 μL, 37% solution in water, 2.41 mmol) and acetic acid (138 μL, 2.41 mmol). The reaction mixture was stirred for 15 minutes, then sodium triacetoxyborohydride (512 mg, 2.41 mmol) was added and stirred for 2 hours at room temperature. The reaction mixture was quenched with saturated aqueous sodium bicarbonate solution, then diluted with dichloromethane. The layers were separated and the aqueous layer was extracted with dichloromethane. The combined organic layers were dried over sodium sulfate. Filtration and concentration provided phenylmethyl N-(3,4-dihydro-2H-pyrano[3,2-b]pyridin-4-yl)-N-methylglycinate (320 mg, 64%) as a clear oil. ¹H NMR (400 MHz, CDCl₃) δ 8.14 (dd, J=3.9, 2.0 Hz, 1H), 7.37-7.32 (m, 5H), 7.10-7.05 (m, 2H), 5.12 (m, 2H), 4.41 (ddd, J=11.1, 8.4, 2.9 Hz, 1H), 4.16 (ddd, J=10.7, 7.2, 3.3 Hz, 1H), 4.04 (t, J=5.8 Hz, 1H), 3.57 (s, 2H), 2.52 (s, 3H), 2.23 (m, 1H), 2.08 (m, 1H); MS m/z 313 (M+H)⁺.

### C)N-(3,4-dihydro-2H-pyrano[3,2-b]pyridin-4-yl)-N-methylglycine

To a solution of phenylmethyl N-(3,4-dihydro-2H-pyrano[3,2-b]pyridin-4-yl)-N-methylglycinate (519 mg, 1.66 mmol) in ethanol (10 mL) was added 10% palladium on carbon (40 mg). The reaction mixture was stirred under a balloon of hydrogen for 4 hours. The reaction mixture was filtered through a pad of celite and rinsed with fresh ethanol. The combined filtrates were concentrated to provide N-(3,4-dihydro-2H-pyrano[3,2-b]pyridin-4-yl)-N-methylglycine (350 mg, 95%) as a clear yellow oil. ¹H NMR (400 MHz, CDCl₃) δ 8.06 (t, J=2.7 Hz, 1H), 7.13-7.12 (m, 2H), 4.71 (dd, J=10.9, 5.8 Hz, 1H), 4.57 (br, 2H), 4.39 (d, J=11.7 Hz, 1H), 4.13 (t, J=11.4 Hz, 1H), 2.67 (s, 3H), 2.37 (d, J=13.5 Hz, 1H), 2.18 (m, 1H); MS m/z 223 (M+H)⁺.

### D) N-[2-(dimethylamino)ethyl]-N-methyl-2-nitro-1,3-benzenediamine

A mixture of 3-chloro-2-nitroaniline (520 mg, 3.01 mmol) and N,N,N′-trimethylethylene diamine (2 mL, 15.4 mmol) was heated in a microwave at 160° C. for 20 minutes. The reaction mixture was cooled and excess amine was removed in vacuo. The residue was chromatographed on silica gel (0 to 7% methanol in dichloromethane) to provide N-[2-(dimethylamino)ethyl]-N-methyl-2-nitro-1,3-benzenediamine (610 mg, 85%) as a red oil. ¹H NMR (400 MHz, CDCl₃) δ 7.06 (dd, J=8.2, 8.2 Hz, 1H), 6.35 (d, J=8.2 Hz, 1H), 6.22 (d, J=8.2 Hz, 1H), 4.86 (br, 2H), 3.20 (t, J=7.4 Hz, 2H), 2.79 (s, 3H), 2.49 (t, J=7.3 Hz, 2H), 2.23 (s, 6H); MS m/z 239 (M+H)⁺.

### E) N¹-[2-(dimethylamino)ethyl]-N¹-methyl-1,2,3-benzenetriamine

To a solution of N-[2-(dimethylamino)ethyl]-N-methyl-2-nitro-1,3-benzenediamine (300 mg, 1.26 mmol) in ethanol (10 mL) was added 10% palladium on carbon (20 mg). The reaction mixture was stirred under a balloon of hydrogen for 2 hours. The reaction mixture was filtered through a pad of celite and rinsed with fresh ethanol. The combined filtrates were concentrated to provide N¹-[2-(dimethylamino)ethyl]-N¹-methyl-1,2,3-benzenetriamine (225 mg, 86%) as a brown oil which was taken on without further purification.

### F) N-(2-{[3,4-dihydro-2H-pyrano[3,2-b]pyridin-4-yl(methyl)amino]methyl}-1H-benzimidazol-4-yl)-N,N,N-trimethyl-1,2-ethanediamine

To a solution of N-(3,4-dihydro-2H-pyrano[3,2-b]pyridin-4-yl)-N-methylglycine (149 mg, 0.672 mmol) in acetonitrile (5 mL) was added N¹-[2-(dimethylamino)ethyl]-N¹-methyl-1,2,3-benzenetriamine (140 mg, 0.672 mmol), diisopropylethyl amine (129 μL, 0.739 mmol), and bis(2-oxo-3-oxazolidinyl)phosphinic chloride (188 mg, 0.739 mmol). The reaction mixture was stirred at room temperature overnight. The reaction mixture was concentrated in vacuo, then partitioned between ethyl acetate and water. The layers were separated and the organic layer was washed with brine, then dried over sodium sulfate. Filtration and concentration provided a crude material which was heated in acetic acid (3 mL) for 1 hour. The acetic acid was removed in vacuo and the resulting residue was partitioned between ethyl acetate and saturated aqueous sodium bicarbonate solution. The organic layer was washed with water and brine, then dried over sodium sulfate. Filtration and concentration, followed by flash chromatography (0 to 10% concentrated aqueous ammonium hydroxide solution in acetonitrile) provided N-(2-{[3,4-dihydro-2H-pyrano[3,2-b]pyridin-4-yl(methyl)amino]methyl}-1H-benzimidazol-4-yl)-N,N,N-trimethyl-1,2-ethanediamine (110 mg, 42%) as a light yellow oil. ¹H NMR (400 MHz, CDCl₃) δ 8.22 (dd, J=2.9, 2.9 Hz, 1H), 7.19-7.12 (m, 3H), 7.07 (dd, J=7.8, 7.8 Hz, 1H), 6.57 (d, J=7.7 Hz, 1H), 4.42 (ddd, J=10.9, 7.7, 3.3 Hz, 1H), 4.17 (ddd, J=10.8, 7.8, 3.1 Hz, 1H), 4.08-3.94 (m, 3H), 3.57 (br, 2H), 2.96 (s, 3H), 2.63 (m, 2H), 2.36 (s, 6H), 2.28 (m, 1H), 2.09 (m, 1H); MS m/z 395 (M+H)⁺.

### Example 2

### 1,1-dimethylethyl 4-(2-{[3,4-dihydro-2H-pyrano[3,2-b]pyridin-4-yl(methyl)amino]methyl}-1H-benzimidazol-4-yl)-1-piperazinecarboxylate (Intermediate)

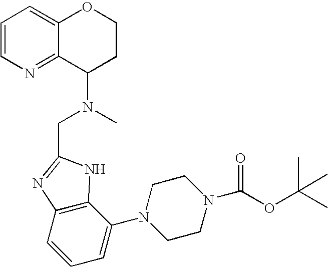

### A) 1,1-Dimethylethyl 4-(3-amino-2-nitrophenyl)-1-piperazinecarboxylate

From 3-chloro-2-nitroaniline (520 mg, 3.01 mmol) and 1-BOC-piperazine (2 g, 10.7 mmol) was obtained 1,1-dimethylethyl 4-(3-amino-2-nitrophenyl)-1-piperazinecarboxylate (600 mg, 62%) as a red oil. ¹H NMR (400 MHz, CDCl₃) δ 7.13 (dd, J=8.1, 8.1 Hz, 1H), 6.44 (d, J=8.4 Hz, 1H), 6.38 (d, J=8.1 Hz, 1H), 4.80 (br, 2H), 3.52 (t, J=4.7 Hz, 4H), 2.93 (t, J=4.1 Hz, 4H), 1.46 (s, 9H); MS m/z 345 (M+Na)⁺.

### B) 1,1-Dimethylethyl 4-(2,3-diaminophenyl)-1-piperazinecarboxylate

From 1,1-dimethylethyl 4-(3-amino-2-nitrophenyl)-1-piperazinecarboxylate (225 mg, 0.698 mmol) and 10% palladium on carbon (20 mg) was obtained 1,1-dimethylethyl 4-(2,3-diaminophenyl)-1-piperazinecarboxylate (204 mg, 100%) as a brown oil which was taken on without further purification.

### C) 1,1-dimethylethyl 4-(2-{[3,4-dihydro-2H-pyrano[3,2-b]pyridin-4-yl(methyl)amino]methyl}-1H-benzimidazol-4-yl)-1-piperazinecarboxylate

From N-(3,4-dihydro-2H-pyrano[3,2-b]pyridin-4-yl)-N-methylglycine (170 mg, 0.767 mmol) and 1,1-dimethylethyl 4-(2,3-diaminophenyl)-1-piperazinecarboxylate (204 mg, 0.698 mmol) was obtained 1,1-dimethylethyl 4-(2-{[3,4-dihydro-2H-pyrano[3,2-b]pyridin-4-yl(methyl)amino]methyl}-1H-benzimidazol-4-yl)-1-piperazinecarboxylate (169 mg, 46%) as a light yellow oil. ¹H NMR (400 MHz, CDCl₃) δ 8.26 (s, 1H), 7.21-7.18 (m, 4H), 7.12 (dd, J=8.0, 8.0 Hz, 1H), 6.65 (br, 1H), 4.45 (m, 1H), 4.21 (m, 1H), 4.07-3.85 (m, 3H), 3.68 (m, 4H), 3.34 (m, 4H), 2.29-2.14 (m, 2H), 1.47 (s, 9H); MS m/z 479 (M+H)⁺.

### Example 3

### N-methyl-N-{[4-(1-piperazinyl)-1H-benzimidazol-2-yl]methyl}-3,4-dihydro-2H-pyrano[3,2-b]pyridin-4-amine

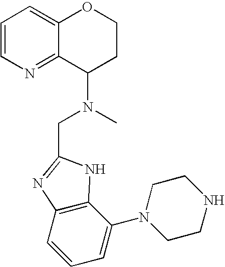

A solution of 1,1-dimethylethyl 4-(2-{[3,4-dihydro-2H-pyrano[3,2-b]pyridin-4-yl(methyl)amino]methyl}-1H-benzimidazol-4-yl)-1-piperazinecarboxylate (169 mg, 0.353 mmol) in 1:1 trifluoroacetic acid:dichloromethane (2 mL) was stirred at room temperature for 1.5 hours. The reaction mixture was concentrated under a stream of nitrogen, then partitioned between ethyl acetate and saturated aqueous sodium bicarbonate solution. The organic layer was washed with brine and dried over sodium sulfate. Filtration and concentration, followed by flash chromatography (0 to 10% concentrated aqueous ammonium hydroxide solution in acetonitrile) provided N-methyl-N-{[4-(1-piperazinyl)-1H-benzimidazol-2-yl]methyl}-3,4-dihydro-2H-pyrano[3,2-b]pyridin-4-amine (63 mg, 47%) as a white foam. ¹H NMR (400 MHz, CDCl₃) δ 8.22 (dd, J=4.1, 1.8 Hz, 1H), 7.26-7.09 (m, 4H), 6.66 (d, J=7.7 Hz, 1H), 5.80 (br, 1H), 4.43 (ddd, J=10.9, 7.1, 3.6 Hz, 1H), 4.18 (ddd, J=11.0, 8.4, 2.6 Hz, 1H), 4.05-3.89 (m, 3H), 3.47 (br, 4H), 3.28 (t, J=4.6 Hz, 4H), 2.32 (s, 3H), 2.26 (m, 1H), 2.11 (m, 1H); MS m/z 379 (M+H)⁺.

### Example 4

### N-methyl-N-{[4-(4-methyl-1-piperazinyl)-1H-benzimidazol-2-yl]methyl}-3,4-dihydro-2H-pyrano[3,2-b]pyridin-4-amine

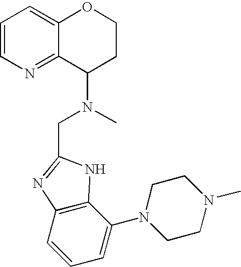

From N-(3,4-dihydro-2H-pyrano[3,2-b]pyridin-4-yl)-N-methylglycine (158 mg, 0.711 mmol) and [2-amino-3-(4-methyl-1-piperazinyl)phenyl]amine (133 mg, 0.645 mmol) was provided N-methyl-N-{[4-(4-methyl-1-piperazinyl)-1H-benzimidazol-2-yl]methyl}-3,4-dihydro-2H-pyrano[3,2-b]pyridin-4-amine (27 mg, 10%) as a clear oil. ¹H NMR (400 MHz, CDCl₃) δ 12.06 (s, 1H), 8.17 (dd, J=3.6, 2.3 Hz, 1H), 7.21-7.18 (m, 2H), 6.98-6.95 (m, 2H), 6.42 (dd, J=5.9, 2.5 Hz, 1H), 4.42 (m, 1H), 4.19 (m, 1H), 3.99 (s, 2H), 3.93 (t, J=6.0 Hz, 1H), 3.45 (br, 4H), 2.51 (br, 4H), 2.30 (m, 1H), 2.24 (s, 3H), 2.23 (s, 3H), 2.05 (m, 1H); MS m/z 393 (M+H)⁺.

### Example 5

### 1,1-dimethylethyl 4-{2-[(3,4-dihydro-2H-pyrano[3,2-b]pyridin-4-ylamino)methyl]-1-methyl-1H-benzimidazol-7-yl}-1-piperazinecarboxylate (Intermediate)

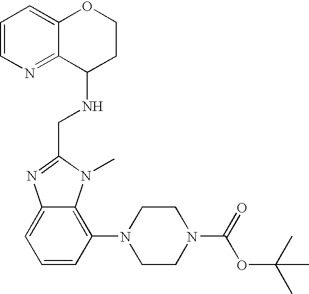

### A) 1,1-dimethylethyl 4-(3-amino-2-nitrophenyl)-1-piperazinecarboxylate

A mixture of 3-chloro-2-nitroaniline (1.00 g, 5.80 mmol) and 1,1-dimethylethyl 1-piperazinecarboxylate (2.00 g, 10.7 mmol) was heated in a microwave at 150° C. for 20 minutes. This reaction was repeated on the same scale. The combined crude reaction mixtures were chromatographed on silica gel (0% to 50% ethyl acetate in hexanes) to provide 1,1-dimethylethyl 4-(3-amino-2-nitrophenyl)-1-piperazinecarboxylate (842 mg, 23%) as a yellow solid. ¹H NMR (400 MHz, CDCl₃) δ 7.12 (app t, J=8.2 Hz, 1H), 6.43 (d, J=8.1 Hz, 1H), 6.38 (d, J=8.0 Hz, 1H), 4.48 (br, 2H), 3.51 (m, 4H), 2.93 (m, 4H), 1.45 (s, 9H).

### B) 1,1-dimethylethyl 4-(2,3-diaminophenyl)-1-piperazinecarboxylate

To a solution of 1,1-dimethylethyl 4-(3-amino-2-nitrophenyl)-1-piperazinecarboxylate (842 mg, 2.61 mmol) in ethanol (40 mL) was added 10% palladium on carbon (100 mg). The reaction mixture was stirred under a hydrogen atmosphere for 2 hours. The reaction mixture was filtered through celite and concentrated. The reaction mixture was taken on crude to the next step.

### C) 1,1-dimethylethyl 4-{2-[({[(phenylmethyl)oxy]carbonyl}amino)methyl]-1H-benzimidazol-4-yl}-1-1piperazinecarboxylate

To a solution of 1,1-dimethylethyl 4-(3-amino-2-nitrophenyl)-1-piperazinecarboxylate (crude from previous step, 2.61 mmol) in acetonitrile was added N-{[(phenylmethyl)oxy]carbonyl}glycine (546 mg, 2.61 mmol), bis(2-oxo-3-oxazolidinyl)phosphinic chloride (998 mg, 3.92 mmol), and N,N-diisopropylethylamine (683 μL, 3.92 mmol). The reaction mixture was stirred at room temperature overnight. The reaction mixture was concentrated in vacuo, then partitioned between ethyl acetate and water. The organic layer was dried over sodium sulfate, filtered and concentrated. The crude material was dissolved in acetic acid (2 mL) and heated at 70° C. for 2 hours. The reaction mixture was concentrated, then diluted with ethyl acetate and washed with saturated aqueous sodium bicarbonate solution and brine. The organic layer was dried over sodium sulfate, filtered and concentrated. The crude material was chromatographed on silica gel (1% to 5% 2M ammonia in methanol/dichloromethane) to provide 1,1-dimethylethyl 4-{2-[({[(phenylmethyl)oxy]carbonyl}amino)methyl]-1H-benzimidazol-4-yl}-1-piperazinecarboxylate (957 mg, 78% yield for 2 steps) as a light red solid. ¹H NMR (400 MHz, CDCl₃) δ 7.33-7.32 (m, 5H), 7.18-7.09 (m, 2H), 6.67 (d, J=7.5 Hz, 1H), 5.89 (br, 1H), 5.13 (s, 2H), 4.57 (d, J=6.2 Hz, 2H), 3.64 (app t, J=4.7 Hz, 4H), 3.32 (br, 4H), 1.49 (s, 9H); MS m/z 466 (M+H)⁺.

### D) 1,1-dimethylethyl 4-{2-[({[(phenylmethyl)oxy]carbonyl}amino)methyl]-1H-benzimidazol-4-yl}-1-piperazinecarboxylate

To a solution of 1,1-dimethylethyl 4-{2-[({[(phenylmethyl)oxy]carbonyl}amino)methyl]-1H-benzimidazol-4-yl}-1-piperazinecarboxylate (957 mg, 2.06 mmol) in N,N-dimethylformamide was added iodomethane (229 μL, 2.47 mmol) and cesium carbonate (1.01 g, 3.09 mmol). The reaction mixture was stirred at room temperature for 2 hours. The reaction mixture was diluted with toluene and washed with water, then brine. The organic layer was dried over sodium sulfate, filtered, and concentrated. The material was chromatographed on silica gel (15% to 100% ethyl acetate in hexanes) to provide 1,1-dimethylethyl 4-{2-[({[(phenylmethyl)oxy]carbonyl}amino)methyl]-1H-benzimidazol-4-yl}-1-piperazinecarboxylate (220 mg, 22%) as a light yellow solid. ¹H NMR (400 MHz, CDCl₃) δ 7.42 (d, J=7.9 Hz, 1H), 7.35-7.29 (m, 5H), 7.11 (app t, J=7.9 Hz, 1H), 6.94 (d, J=7.8 Hz, 1H), 6.26 (br, 1H), 5.12 (s, 2H), 4.61 (d, J=5.3 Hz, 2H), 4.11-4.08 (m, 5H), 3.03 (m, 4H), 2.81 (m, 2H), 1.48 (s, 9H); MS m/z 480 (M+H)⁺.

### E) 1,1-dimethylethyl 4-[2-(aminomethyl)-1H-benzimidazol-4-yl]-1-piperazinecarboxylate

To a solution of 1,1-dimethylethyl 4-{2-[({[(phenylmethyl)oxy]carbonyl}amino)methyl]-1H-benzimidazol-4-yl}-1-piperazinecarboxylate (220 mg, 0.489 mmol) in ethanol (6 mL) was added 10% palladium on carbon (40 mg). The reaction mixture was stirred under a balloon of hydrogen overnight. The reaction mixture was diluted with ethanol and filtered through celite to remove the catalyst. The filtrate was concentrated to provide 153 mg of a crude material which was taken on to the next step.

### F) 1,1-dimethylethyl 4-{2-[(3,4-dihydro-2H-pyrano[3,2-b]pyridin-4-ylamino)methyl]-1-methyl-1H-benzimidazol-7-yl}-1-piperazinecarboxylate

To a solution of 1,1-dimethylethyl 4-[2-(aminomethyl)-1H-benzimidazol-4-yl]-1-piperazinecarboxylate (153 mg, 0.443 mmol) and 2,3-dihydro-4H-pyrano[3,2-b]pyridin-4-one (66 mg, 0.443 mmol) in 1,2 dichloroethane (2 mL) was added acetic acid (39 μL, 0.67 mmol). The reaction mixture was stirred 2 hours at room temperature. Sodium triacetoxyborohydride (188 mg, 0.886 mmol) was added and stirred overnight at room temperature. The reaction mixture was diluted with dichloromethane and washed with saturated aqueous sodium bicarbonate solution. The organic layer was dried over sodium sulfate. Filtration and concentration, followed by flash chromatography (0% to 10% aqueous ammonium hydroxide in acetonitrile) to provide 1,1-dimethylethyl 4-{2-[(3,4-dihydro-2H-pyrano[3,2-b]pyridin-4-ylamino)methyl]-1-methyl-1H-benzimidazol-7-yl}-1-piperazinecarboxylate (208 mg, 98%) as a yellow oil. ¹H NMR (400 MHz, CDCl₃) δ 8.13 (m, 1H), 7.48 (d, J=8.1 Hz, 1H), 7.16-7.07 (m, 3H), 6.95 (d, J=7.7 Hz, 1H), 4.39-4.01 (m, 10H), 3.10 (m, 4H), 2.85 (m, 2H), 2.60-2.15 (m, 2H), 1.49 (s, 9H); MS m/z 479 (M+H)⁺.

### Example 6

### N-{[1-methyl-7-(1-piperazinyl)-1H-benzimidazol-2-yl]methyl}-3,4-dihydro-2H-pyrano[3,2-b]pyridin-4-amine (Intermediate)

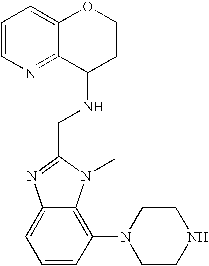

To a solution of 1,1-dimethylethyl 4-{2-[(3,4-dihydro-2H-pyrano[3,2-b]pyridin-4-ylamino)methyl]-1-methyl-1H-benzimidazol-7-yl}-1-piperazinecarboxylate (208 mg, 0.435 mmol) in dichloromethane (1.5 mL) was added 50:50 solution of trifluoroacetic acid in dichloromethane (3 mL). The reaction mixture was stirred at room temperature 1.5 hours. The reaction mixture was concentrated under a stream of nitrogen. The residue was taken up in methanol and solid sodium bicarbonate was added. The solids were removed by filtration and the filtrate was concentrated in vacuo to provide N-{[1-methyl-7-(1-piperazinyl)-1H-benzimidazol-2-yl]methyl}-3,4-dihydro-2H-pyrano[3,2-b]pyridin-4-amine (163 mg, 99%) as an orange oil. ¹H NMR (400 MHz, CD₃OD) δ 8.22 (d, J=15.3 Hz, 1H), 7.57-7.09 (m, 5H), 4.86-4.13 (m, 8H), 3.52-3.15 (m, 8H), 2.68-2.24 (m, 2H); MS m/z 379 (M+H)⁺.

### Example 7

### N-methyl-N-{[1-methyl-7-(4-methyl-1-piperazinyl)-1H-benzimidazol-2-yl]methyl}-3,4-dihydro-2H-pyrano[3,2-b]pyridin-4-amine

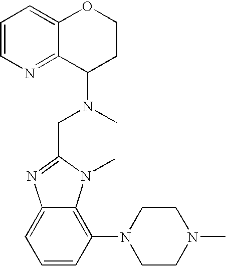

To a solution of N-{[1-methyl-7-(1-piperazinyl)-1H-benzimidazol-2-yl]methyl}-3,4-dihydro-2H-pyrano[3,2-b]pyridin-4-amine (163 mg, 0.431 mmol) in 1,2 dichloroethane (3 mL) was added formaldehyde (171 μL, 37% solution in water, 2.11 mmol) and acetic acid (121 μL, 2.11 mmol). The reaction mixture was stirred at room temperature 1 hour. Sodium triacetoxyborohydride (224 mg, 1.06 mmol) was added and stirred 1 hour at room temperature. The reaction mixture was diluted with dichloromethane and washed with saturated aqueous sodium bicarbonate solution. The organic layer was dried over sodium sulfate. Filtration and concentration, followed by flash chromatography (2% to 7% aqueous ammonium hydroxide in acetonitrile) provided N-methyl-N-{[1-methyl-7-(4-methyl-1-piperazinyl)-1H-benzimidazol-2-yl]methyl}-3,4-dihydro-2H-pyrano[3,2-b]pyridin-4-amine (10 mg, 6%) as a light yellow oil. ¹H NMR (400 MHz, CDCl₃) δ 8.22 (m, 1H), 7.47 (d, J=8.0 Hz, 1H), 7.15-7.09 (m, 3H), 6.99 (d, J=7.7 Hz, 1H), 4.40 (m, 1H), 4.23-3.89 (m, 7H), 3.14 (br, 6H), 2.59-2.48 (m, 5H), 2.40-2.11 (m, 5H); MS m/z 407 (M+H)⁺.

### Biological Section

### Fusion Assay

### Plasmid Generation

The complete coding sequences of HIV-1 tat (GenBank Accession No. X07861) and rev (GenBank Accession No. M34378) were cloned into pcDNA3.1 expression vectors containing G418 and hygromycin resistance genes, respectively. The complete coding sequence of the HIV-1 (HXB2 strain) gp160 envelope gene (nucleotide bases 6225-8795 of GenBank Accession No. K03455) was cloned into plasmid pCRII-TOPO. The three HIV genes were additionally inserted into the baculovirus shuttle vector, pFastBacMam1, under the transcriptional control of the CMV promoter. A construction of the pHIV-I LTR containing mutated NFkB sequences linked to the luciferase reporter gene was prepared by digesting pcDNA3.1, containing the G418 resistance gene, with Nru I and Bam HI to remove the CMV promoter. LTR-luc was then cloned into the Nru I/Bam HI sites of the plasmid vector. Plasmid preparations were performed after the plasmids were amplified in Escherichia coli strain DH5-alpha. The fidelity of the inserted sequences was confirmed by double-strand nucleotide sequencing using an ABI Prism Model 377 automated sequencer.

### BacMam Baculovirus Generation

Recombinant BacMam baculoviruses were constructed from pFastBacMam shuttle plasmids by using the bacterial cell-based Bac-to-Bac system. Viruses were propagated in Sf9 (Spodoptera frugiperda) cells cultured in Hink's TNM-FH Insect media supplemented with 10% (v/v) fetal bovine serum and 0.1% (v/v) pluronic F-68 according to established protocols.

### Cell Culture

Human osteosarcoma (HOS) cells that naturally express human CXCR4 were transfected with human CCR5, human CD4 and the pHIV-LTR-luciferase plasmid using FuGENE 6 transfection reagent. Single cells were isolated and grown under selection condition in order to generate a stable HOS (hCXCR4/hCCR5/hCD4/pHIV-LTR-luciferase) clonal cell line. The cells were maintained in Dulbeccos modified Eagles media supplemented with 10% fetal calf serum (FCS), G418 (400 ug/ml), puromycin (1 ug/ml), mycophenolic acid (40 ug/ml), xanthine (250 ug/ml) and hypoxanthine (13.5 ug/ml) to maintain a selection pressure for cells expressing the LTR-luciferase, hCCR5 and hCD4, respectively. Human embryonic kidney (HEK-293) cells stably transfected to express the human macrophage scavenging receptor (Class A, type 1; GenBank Accession No. D90187), were maintained in DMEM/F-12 media (1:1) supplemented with 10% FCS and 1.5 ug/ml puromycin. The expression of this receptor by the HEK-293 cells enhances their ability to stick to tissue culture treated plasticware.

### Transduction of HEK-293 Cells

HEK-293 cells were harvested using enzyme-free cell dissociation buffer. The cells were resuspended in DMEM/F-12 media supplemented with 10% FCS and 1.5 ug/ml and counted. Tranductions were performed by direct addition of BacMam baculovirus containing insect cell media to cells. The cells were simultaneously transduced with BacMam baculovirus expressing HIV-1 tat, HIV-1 rev and HIV-1 gp160 (from the HXB2 HIV strain). Routinely an MOI of 10 of each virus was added to the media containing the cells. 2 mM butyric acid was also added to the cells at this stage to increase protein expression in transduced cells. The cells were subsequently mixed and seeded into a flask at 30 million cells per T225. The cells were incubated at 37° C., 5% CO₂, 95% humidity for 24 h to allow for protein expression.

### Cell/Cell Fusion Assay Format

HEK and HOS cells were harvested in DMEM/F-12 media containing 2% FCS and DMEM media containing 2% FOS, respectively, with no selection agents added. Compounds were plated as 1 ul spots in 100% DMSO on a 96-well CulturPlate plates. HOS cells (50 ul) were added first to the wells, followed immediately by the HEK cells (50 ul). The final concentration of each cell type was 20,000 cells per well. Following these additions, the cells were returned to a tissue culture incubator (37° C.; 5% CO₂/95% air) for an additional 24 h.

### Measurement of Luciferase Production

Following the 24 h incubation, total cellular luciferase activity was measured using the LucLite Plus assay kit (Packard, Meridien, Conn.). In brief, 100 ul of this reagent was added to each well. The plates were sealed and mixed. The plates were dark adapted for approximately 10 min prior to the luminescence being read on a Packard TopCount.

### Functional Assay

### Cell Culture

Human embryonic kidney (HEK-293) cells were maintained and harvested as described above. Cells were plated in 96-well, black clear bottom, poly-lysine coated plates at a concentration of 40,000 cells per well in a final volume of 100 ul containing human CXCR4BacMam (MOI=25) and Gqi5 BacMam (MOI=12.5). The cells were incubated at 37° C., 5% CO₂, 95% humidity for 24 h to allow for protein expression.

### Functional FLIPR Assay

After the required incubation time the cells were washed once with 50 ul of fresh serum-free DMEM/F12 media containing probenicid. 50 ul of dye solution was then added to the cells (Calcium Plus Assay Kit Dye; Molecular Devices) was dissolved in 200 ml of the above probenicid/BSA containing media and incubated for 1 h. Cell plates were transferred to a Fluorometric Imaging Plate Reader (FLIPR). Upon addition the effect of the compounds on the change in [Ca²⁺], was examined to determine if the compounds were agonists or antagonists (ability to block SDF-1 alpha activity) at the CXCR4 receptor. IC₅₀ values are determined and pKb values are calculated using the Leff and Dougall equation: K_{B}=IC₅₀/((2+([agonist]/EC₅₀̂b)̂1/b−1) Where IC₅₀ is that defined by the antagonist concentration-response curve [agonist] is the EC₈₀ concentration of agonist used EC₅₀ is that defined by the agonist concentration-response curve b is the slope of the agonist concentration-response curve.

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24 h.

Subsequently, 50 ul of the desired drug solution (4 times the final concentration) was added to each well and the plates were returned to the tissue culture incubator (37° C.; 5% CO₂/95% air) for 1 h. Following this incubation 50 ul of diluted virus was added to each well (approximately 2 million RLU per well of virus). The plates were returned to the tissue culture incubator (37° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

Compounds of the present invention have anti-HIV activity in the range IC₅₀=1-1000 nM. Moreover, compounds of the present invention are believed to provide a desired pK profile. Also, compounds of the present invention are believed to provide a desired selectivity, such as not promoting an undesired protein shift.

Test compounds were employed in free or salt form.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I): 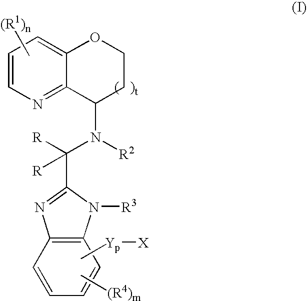
wherein:
t is 1, or 2;
each R independently is H, alkyl, alkenyl, alkynyl, haloalkyl, cycloalkyl, —R^{a}Ay, —R^{a}OR⁵, or —R^{a}S(O)_{q}R⁵;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl R^{a}Ay, R^{a}OR⁵, —R^{a}S(O)_{q}R⁵;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido; m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, S(O)_{q}NR¹⁰—, or —NR¹⁰S(O)_{q}—;
X is —N(R¹⁰)₂, -AyN(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, alkyl, cycloalkyl, alkenyl, alkynyl, cycloalkenyl, —R^{a}Cycloalkyl —R^{a}OH, —R^{a}OR⁵, —R^{a}NR⁶R⁷, or —R^{a}Het, provided that when p is 0 and X is —N(R¹⁰)₂, then R¹⁰ is not H;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁸, —R^{a}NR⁸R⁹, -Ay, -Het, —R^{a}Ay —R^{a}Het, or —S(O)_{q}R¹⁰;
each of R⁸ and R⁹ independently are selected from H or alkyl;
each q independently is 0, 1, or 2;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or a pharmaceutically acceptable salt thereof.

2. A compound of claim 1 wherein t is 1.

3-4. (canceled)

5. A compound of claim 1 wherein n is 0.

6. A compound of claim 1 wherein n is 1; R¹ is halogen, haloalkyl, alkyl, OR¹⁰, NR⁶R⁷, CO₂R¹⁰, CONR⁶R⁷, or cyano.

7. The compound of claim 1 wherein R² is H, alkyl, haloalkyl, or cycloalkyl.

8. (canceled)

9. A compound of claim 1 wherein R³ is H, alkyl, haloalkyl, cycloalkyl, alkenyl, or alkynyl.

10-11. (canceled)

12. The compound of claim 1 wherein m is 0.

13. A compound of claim 1 wherein m is 1 or 2.

14. (canceled)

15. A compound of claim 1, wherein m is 1 and R⁴ is halogen, haloalkyl, alkyl, OR¹⁰, NR⁶R⁷, CO₂R¹⁰, CONR⁶R⁷, or cyano.

16. A compound of claim 1 wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

17. (canceled)

18. A compound of claim 1 wherein
p is 1;
Y is —N(R¹⁰)—, —O—, —S—, —CONR¹⁰—, —NR¹⁰CO—, or —S(O)_{q}NR¹⁰—; and
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

19. (canceled)

20. A compound of formula (I-A): 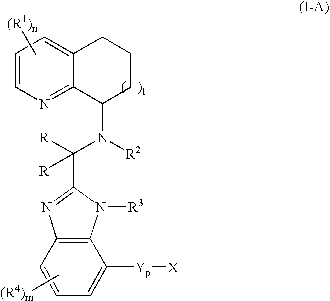
wherein:
t is 1, or 2;
each R independently is H, alkyl, alkenyl, alkynyl, haloalkyl, cycloalkyl, —R^{a}Ay, —R^{a}OR⁵, or —R^{a}S(O)_{q}R⁵;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het, provided that when p is 0 and X is —N(R¹⁰)₂, then R¹⁰ is not H;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁸, —R^{a}NR⁸R⁹, -Ay, -Het, —R^{a}Ay —R^{a}Het, or —S(O)_{q}R¹⁰;
each of R⁸ and R⁹ independently are selected from H or alkyl;
each q independently is 0, 1, or 2;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or a pharmaceutically acceptable salt thereof.

21. A compound of formula (I-B): 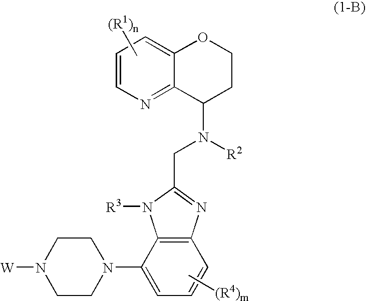
wherein R¹ is halogen, C₁-C₈haloalkyl, C₁-C₈ alkyl, OR¹⁰, NR⁶R⁷, CO₂R¹⁰, CONR⁶R⁷₉ or cyano;
n is 0, 1; or 2;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
each R^{a} independently is alkylene, cycloalkylene, alkenylene, cycloalkenylene, or alkynylene;
m is 0, 1, or 2;
R⁴ is one or more of halogen, C₁-C₈haloalkyl, C₁-C₈ alkyl, OR¹⁰, NR⁶R⁷, CO₂R¹⁰, CONR⁶R⁷, or cyano;
each R¹⁰, independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷ or —R^{a}Het;
each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each R^{a} independently is C₁-C₈ alkylene, C₃-C₇ cycloalkylene, C₂-C₆ alkenylene, C₃-C₇ cycloalkenylene, or C₂-C₆ alkynylene;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
W is H or C₁-C₈ alkyl;
or a pharmaceutically acceptable salt thereof.

22. A compound of formula (I-C): 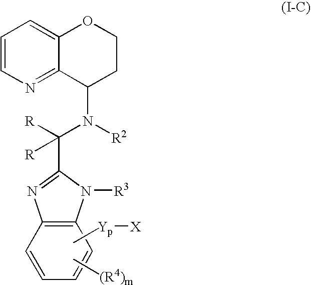
wherein each R is H or C₁-C₈ alkyl;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
m is 0, 1, or 2;
R⁴ is one or more of halogen, C₁-C₈haloalkyl, C₁-C₈ alkyl, OR¹⁰, NR⁶R⁷, CO₂R¹⁰, CONR⁶R⁷, or cyano;
p is 0 or 1;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂;
Y is —N(R¹⁰)—, —O—, —S—, —CONR¹⁰—, —NR¹⁰CO—, or —S(O)_{q}NR¹⁰—;
each q independently is 0, 1, or 2;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷, or R^{a}Het;
each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each R^{a} independently is C₁-C₈ alkylene, C₃-C₇ cycloalkylene, C₂-C₆ alkenylene, C₃-C₇ cycloalkenylene, or C₂-C₆ alkynylene;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or a pharmaceutically acceptable salt thereof.

23. A compound of formula (I-D): 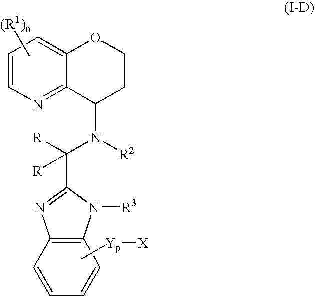
wherein each R is H or C₁-C₈ alkyl;
R¹ is halogen, C₁-C₈haloalkyl, C₁-C₈ alkyl, OR¹⁰, NR⁶R⁷, CO₂R¹⁰, CONR⁶R⁷, or cyano;
n is 0, 1; or 2;
R is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
p is 0 or 1;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂;
Y is —N(R¹⁰)—, —O—, —S—, —CONR¹⁰—, —NR¹⁰CO—, or —S(O)_{q}NR¹⁰—;
each q independently is 0, 1, or 2;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆ alkynyl, C₃-C₇ cycloalkenyl, —R^{a}cycloalkyl, —R^{a}OH, —R^{a}OR⁵, —R^{a}NR⁶R⁷, or —R^{a}Het;
each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or a pharmaceutically acceptable salt thereof.

24. A compound of formula (I-E): 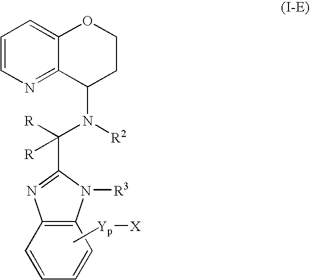
wherein each R is H or C₁-C₈ alkyl;
R² is H, C₁-C₈ alkyl, C₁-C₈haloalkyl or C₃-C₇cycloalkly;
R³ is H, C₁-C₈ alkyl, C₁-C₈haloalkyl, C₃-C₇ cycloalkyl, C₂-C₆ alkenyl, or C₂-C₆ alkynyl;
p is 0 or 1;
X is R^{a}N(R¹⁰)₂, AyR^{a}N(R¹⁰)₂, —R^{a}AyR^{a}N(R¹⁰)₂, -Het, R^{a}Het, -HetN(R¹⁰)₂, —R^{a}HetN(R¹⁰)₂, or -HetR^{a}N(R¹⁰)₂;
Y is —N(R¹⁰)—, —O—, —S—, —CONR¹⁰—, —NR¹⁰CO—, or —S(O)_{q}NR¹⁰—;
each q independently is 0, 1, or 2;
each R^{a} independently is C₁-C₈ alkylene, C₃-C₇ cycloalkylene, C₂-C₆ alkenylene, C₃-C₇ cycloalkenylene, or C₂-C₆ alkynylene;
each R¹⁰ independently is H, C₁-C₈ alkyl, C₃-C₇ cycloalkyl, C₂-C₆ alkenyl, C₂-C₆alkynyl, C₃-C₇ cycloalkenyl, —R^{a}cycloalkyl, —R^{a}OH, —R^{a}OR⁵, —R^{a}NR⁶R⁷, or —R^{a}Het;
each R⁵ independently is H, C₁-C₈ alkyl, C₂-C₆ alkenyl, C₂-C₆ alkynyl, C₃-C₇ cycloalkyl, or -Ay;
each of R⁶ and R⁷ independently are selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁸, —R^{a}NR⁸R⁹, -Ay, -Het, —R^{a}Ay, —R^{a}Het, or —S(O)_{q}R¹⁰;
each of R⁸ and R⁹ independently are selected from H or C₁-C₈ alkyl;
each Ay independently represents an aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
each Het independently represents a heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₇ cycloalkyl, C₃-C₇ cycloalkoxy, cyano, amide, amino, and C₁-C₈ alkylamino;
or a pharmaceutically acceptable salt thereof.

25. A compound of selected from the group consisting of:
N-(2-{[3,4-dihydro-2H-pyrano[3,2-b]pyridin-4-yl(methyl)amino]methyl}-1H-benzimidazol-4-yl)-N,N′,N′-trimethyl-1,2-ethanediamine;
N-methyl-N-{[4-(1-piperazinyl)-1H-benzimidazol-2-yl]methyl}-3,4-dihydro-2H-pyrano[3,2-b]pyridin-4-amine;
N-methyl-N-{[4-(4-methyl-1-piperazinyl)-1H-benzimidazol-2-yl]methyl}-3,4-dihydro-2H-pyrano[3,2-b]pyridin-4-amine; and
N-methyl-N-{[1-methyl-7-(4-methyl-1-piperazinyl)-1H-benzimidazol-2-yl]methyl}-3,4-dihydro-2H-pyrano[3,2-b]pyridin-4-amine; or a pharmaceutically acceptable salt thereof.

26. (canceled)

27. A pharmaceutical composition comprising a compound according to claim 1, and a pharmaceutically acceptable carrier.

28-42. (canceled)
